(19) United States
(12) Patent Application Publication
(10) Pub. No.: US 2006/0216279 A1
(43) Pub. Date: Sep. 28, 2006

(54) Myostatin inhibiting fusion polypeptides and therapeutic methods thereof

(76) Inventors: David J. Glass, Cambridge, MA (US); Brian A. Clarke, Cambridge, MA (US)

Correspondence Address: REGENERON PHARMACEUTICALS, INC, 777 OLD SAW MILL RIVER ROAD, TARRYTOWN, NY, 10591, US

(21) Appl. No.: 11/388,304

(22) Filed: Mar. 22, 2006

Related U.S. Application Data
(60) Provisional application No. 60/664,111, filed on Mar. 22, 2005.
(60) Provisional application No. 60/680,569, filed on May 13, 2005.
(60) Provisional application No. 60/692,177, filed on Jun. 20, 2005.
(60) Provisional application No. 60/724,471, filed on Oct. 7, 2005.

(51) Int. Cl. A61K 38/54; C07H 21/04; C12N 9/99; C12P 21/06; C12N 1/21; C12N 5/06

(52) U.S. Cl. 424/94.2; 435/69.1; 435/184; 435/252.31; 435/252.33; 435/358; 514/12; 536/23.2

(57) ABSTRACT

Myostatin inhibiting GDF8 and GDF11 fusion polypeptides and their encoding nucleic acids are disclosed. Pharmaceutical compositions comprising these fusion polypeptides or their corresponding nucleic acids and methods of use are also disclosed.

### CROSS-REFERENCE TO RELATED APPLICATIONS

This application claims the benefit under 35 USC § 119(e) of U.S. Provisional 60/664,111 filed 22 Mar. 2005, U.S. Provisional 60/680,569 filed 13 May 2005, U.S. Provisional 60/692,177 filed 20 Jun. 2005, and U.S. Provisional 60/724,471 filed 7 Oct. 2005, which applications are herein specifically incorporated by reference in their entirety.

### BACKGROUND OF THE INVENTION

1. Field of the Invention

This invention relates to nucleic acid and amino acid compositions comprising inhibitors of myostatin GDF8 or GDF11, and methods of using these inhibitors for treatment of conditions related to myostatin.

2. Statement Regarding Related Art

Myostatin is a member of the transforming growth factor β (TGF-β) family expressed almost exclusively in skeletal muscle. Myostatin, also termed “Growth and Differentiation Factor-8” (GDF8), is produced as a precursor protein that contains a signal sequence, an N-terminal propeptide domain, and a C-terminal domain that is the active ligand (Hill et al. (2002) J. Biol. Chem. 277:40735-40741). Proteolytic processing between the propeptide domain and the C-terminal domain releases the mature myostatin protein. The myostatin propeptide is known to bind and inhibit myostatin in vitro and in vivo. Fragments of the myostatin propeptide have been characterized (Jiang et al. (2004) Biochem Biophys Res Comm 315 (2004) 525-531); certain fragments have been shown to maintain binding and inhibition of myostatin, whereas other fragments have been stated to be inactive. Wolfman et al. (U.S. patent publication 2003/0104406) have prepared modified GDF8 and BMP-11 propeptides and fusion proteins that form inactive complexes with GDF or BMP proteins and that demonstrate a greater in vivo half-life compared to unmodified propeptides.

### SUMMARY OF THE INVENTION

The present invention rests in part on the identification of truncated GDF8 and GDF11 prodomain fragments that are useful to bind to and inhibit myostatin. The identification of these active smaller fragments allows for ease of production and potentially lower toxicity and antigenicity. Furthermore, the smaller size of the active fragments allows for production of fusion polypeptides that may have improved efficacy, as well as a longer half-life. Accordingly, in its broadest aspect, the invention provides a myostatin inhibiting fusion polypeptide, comprising a GDF8 or a GDF11 prodomain fragment, or variable thereof, capable of binding and inhibiting the biological activity of myostatin.

In a first aspect, the invention provides an isolated nucleic acid molecule encoding a myostatin-inhibiting fusion polypeptide (P)ₓ−M, wherin P is a fragment or variant of a growth and differentiation factor (GDF) prodomain, M is a multimerizing component, and wherein X is a number between 1 and 10. In various embodiments, P is a fragment or variant of a GDF8 or GDF11 prodomain.

In one embodiment, P is a fragment or variant of a GDF8 prodomain (SEQ ID NO:1) having a deletion of at least about 165 to at least about 176 amino acids from the carboxy terminal of SEQ ID NO:1, M is a multimerizing component, and X is a number between 1 and 10. The invention further comprises a variant nucleic acid molecule encoding a GDF8 prodomain fragment having at least 90% identity to SEQ ID NO:1. The nucleic acid molecule of the invention may optionally encode a signal sequence component, for example, that shown in SEQ ID NO:22.

In one embodiment, the GDF8 prodomain fragment is the GDF8 prodomain of SEQ ID NO:1 having a carboxy terminal deletion of at least about 165 amino acids, at least about 171 amino acids, or at least about 176 amino acids, optionally comprising an amino terminal deletion of about 29 amino acids. In a particular embodiment, the GDF8 prodomain fragment is amino acids 1-75 of the native GDF8 prodomain sequence (SEQ ID NO:1). In other embodiments, the GDF8 prodomain fragment is one of SEQ ID NO:3, 4, 5, 6, 7, or 8. In specific embodiments, one or more Cys residues are changed to a different amino acid residue to inhibit protein aggregation upon expression. In specific embodiments, one or more of the Cys residues at position 39, 42 and/or 76 are changed to a different amino acid. In a specific embodiment, the Cys residue is changed to either Ala or Ser. Examples of these variant constructs include, but are not limited to, hGDF8(1-105)-hFc, wherein the Cys at position 76 is replaced with Ala, for example SEQ ID NO:14 additionally having C39S and C42S. In various embodiments, amino acids may be deleted from the GDF8 prodomain fragment, for example, amino acids at positions 39-42 of SEQ ID NO:1 may be deleted.

In another embodiment, P is a fragment or variant of a GDF11 prodomain (SEQ ID NO: 25) having a deletion of at least about 173 to at least about 184 amino acids from the carboxy terminal of SEQ ID NO:25, optionally further comprising an amino terminal deletion of about 30 amino acids, M is a multimerizing component, and X is a number between 1 and 10. The invention further comprises a nucleic acid molecule encoding a GDF11 prodomain fragment having at least 90% identity to SEQ ID NO: 25. The nucleic acid molecule of the invention may optionally encode a signal sequence component, for example, that shown in SEQ ID NO:40.

In particular embodiments, the human GDF11 prodomain fragment is amino acids 1-77 (SEQ ID NO:27), or 1-71 (SEQ ID NO:28) or 1-66 (SEQ ID NO:29) of the native GDF11 prodomain sequence, optionally comprising a deletion of amino acids 1-30 (SEQ ID NO:30, 31, 32). In specific embodiments, Cys residues may be modified to a different amino acid, e.g., to Ser or Ala, or one or more amino acids residues may be deleted.

The fusion polypeptide of the invention may comprise a single P component, or may comprise multiple P components. Preferably, X is 1-10; more preferably X is 1-5, more preferably X is 1-3, and most preferably, X is 1 or 2. In specific embodiments, two or three P components may be used to increase cooperativity and/or avidity. The fusion polypeptides of the invention are capable of specifically binding myostatin with an affinity of at least 10⁻⁷ M, more preferably at least 10⁻⁸ M, as determined by assay methods known in the art, for example, BiaCore analysis. By the term “affinity” is meant the equilibrium dissociation constant. Further, the components of may be arranged in a variety of configurations, for example, P-M, P—P-M, P-M-P, P—P-M-P—P, etc.

When the embodiment comprises multiple P components, the components may be connected directly to each other or connected via one or more spacer sequences. In one preferred embodiment, the components are fused directly to each other. In another preferred embodiment, the components are connected with a nucleic acid sequence encoding a spacer of about 1-200 amino acids. Any spacer known to the art may be used to connect the polypeptide components.

In a further embodiment, P may encompass a variant of the above GDF8 or GDF11 propeptide fragments which differ from the naturally occuring sequence by having one or more deletions, mutations, or insertions, provided that the resulting variant is capable of specifically binding to myostatin, for example, variants having a modification or deletion of one or more Cys residues. The variants may have a nucleic acid sequence about 90%, 95%, 99% or greater identity to a native propeptide sequence.

The multimerizing component (M) includes any natural or synthetic sequence capable of interacting with another multimerizing component to form a higher order structure, e.g., a dimer, a trimer, etc. In a particular embodiment, M may be selected from the group consisting of (i) an immunoglobulin-derived domain, (ii) an amino acid sequence between 1 to about 500 amino acids in length, optionally comprising at least one cysteine residue, (iii) a leucine zipper, (iv) a helix loop motif, and (v) a coil-coil motif. In one embodiment, M comprises an immunoglobulin-derived domain from, for example, human IgG, IgM or IgA. In a more particular embodiment, the immunoglobulin-derived domain may be selected from the group consisting of the Fc domain of IgG or the heavy chain of IgG. The Fc domain of IgG may be selected from the isotypes consisting of IgG1, IgG2, IgG3, and IgG4, as well as any allotype within each isotype group.

In yet another particular embodiment, the nucleic acid encoding fusion polypeptide of the invention may further comprise a targeting ligand, or derivative or fragment thereof, which is capable of binding specifically to a pre-selected cell surface protein, thereby delivering the GDF8 or GDF11 propeptide fragment to a target cell, e.g. a muscle cell. In a more particular embodiment, the targeting component is MuSK ligand, or a fragment of a MuSK ligand capable of binding the MuSK receptor. In a more particular embodiment, the MuSK-specific ligand is agrin or a fragment or derivative thereof capable of binding MuSK, or an anti-MuSK antibody or fragment or derivative thereof, including, for example, a single chain antibody fragment (such as an scFv). In a further particular embodiment, the targeting protein is a muscle-targeting protein which binds the MuSK receptor having the amino acid sequence of human agrin (SEQ ID NO:21), or a variant thereof capable of binding the MuSK receptor. Fragments of agrin useful in the invention include fragments comprising at least about 200 amino acids of the C-terminal sequence of SEQ ID NO:21, including about 300-492, about 260-492, about 222 to 492, about 178-492, about 76-492, and about 60-492 of SEQ ID NO:21. In addition, further amino terminal deletions from these fragments may be prepared. In another particular embodiment, the muscle-targeting ligand of the muscle-targeting fusion polypeptide comprises three or more muscle cadherin (Shimoyama et al. (1998) J. Biol. Chem. 273(16): 10011-10018; Shibata et al. (1997) J. Biol. Chem. 272(8):5236-5270) extracellular cadherin domains, or derivatives or fragments thereof, capable of binding specifically to a muscle cell or other cell that expresses homophilic muscle cadherins. In one particular embodiment, the muscle-targeting ligand consists essentially of the first three (3) or four (4) N-terminal extracellular domains of M-cadherin (SEQ ID NO:11).

In a second aspect, the invention provides a myostatin-inhibiting fusion polypeptide (P)ₓ−M, wherein P, M, and X are as described above. In specific embodiments, the fusion polypeptide of the invention is exemplified by the amino acid sequence of SEQ ID NO:12-19 and 33-40.

In a third aspect, the invention features a multimeric protein, comprising two or more fusion polypeptides of the invention. In a specific embodiment, the multimeric protein is a dimer. The dimeric myostatin-binding protein of the invention are capable of binding myostatin with an affinity of at least 10⁻⁷ M, as determined by assay methods known in the art.

A fourth aspect of the invention provides a vector comprising a nucleic acid molecule of the invention. In one particular embodiment, the invention provides a vector comprising the nucleic acid molecules of the invention, including expression vectors comprising a nucleic acid molecule operatively linked to an expression control sequence.

A fifth aspect of the invention provides host-vector systems for the production of a fusion polypeptide which comprise the expression vector in a suitable host cell. The host cell may be selected from the group consisting of without limitation, a bacterial cell, a yeast cell, an insect cell, and a mammalian cell. Examples of suitable cells include E. coli, B. subtilis, BHK, COS and CHO cells. In yet another particular embodiment, the fusion polypeptides of the invention may be modified by acetylation or pegylation. Methods for acetylating or pegylating a protein are well known in the art.

A sixth aspect of the the invention provides a method of producing a myostatin-inhibiting fusion polypeptide of the invention, comprising culturing a host cell transfected with a vector comprising a nucleic acid molecule of the invention, under conditions suitable for expression of the protein from the host cell, and recovering the polypeptide so produced.

A seventh aspect of the invention provides pharmaceutical compositions comprising a myostatin-inhibiting fusion polypeptide of the invention with a pharmaceutically acceptable carrier. Such pharmaceutical compositions may comprise the fusion proteins, multimers, or nucleic acids which encode them.

In an eighth aspect, the invention provides therapeutic methods for the treatment of a disease or condition, comprising administering a therapeutically effective amount of a myostatin-inhibiting fusion polypeptide of the invention to a subject in need thereof, or a subject at risk for development of that disease or condition. When the disease or condition is a muscle condition, such as atrophy, the therapeutic method of the invention comprises administering a therapeutically effective amount of a muscle-targeting GDF8 or GDF11 fusion polypeptide of the invention to a subject in need thereof, wherein the muscle-related disease or condition is ameliorated or inhibited. In particular embodiments, the invention features a method of inhibiting or ameliorating muscle atrophy, comprising administering a therapeutically effective amount of a fusion polypeptide comprising myostatin propeptide and one of agrin or Fc. In yet another embodiment, the myostatin-inhibiting fusion polypeptide of the invention is used to induce hypertrophy or regeneration of muscle.

The muscle-related condition or disorder treated by the fusion polypeptides of the invention may arise from a number of sources, including for example: aging, denervation, casting, inactivity (disuse), bed rest, congestive heart failure, diabetes, renal failure, growth hormone deficiency, IGF1-deficiency, immobilization, inflammation, such as in chronic inflammatory conditions such as rheumatoid arthritis, mechanic ventilation (resulting in atrophy of the diaphragm), renal failure, sarcopenia, sepsis-induced cachexia, glucocorticoid-induced atrophy, cytokine-induced atrophy settings, sepsis-induced atrophy, cachexia associated with AIDS, cachexia associated with cancer, cachexia associated with burns, degenerative neuropathy, metabolic neuropathy, inflammatory neuropathy, spinal muscular atrophy, autoimmune motor neuropathy and muscular dystrophy.

Other objects and advantages will become apparent from a review of the ensuing detailed description and attendant claims taken in conjunction with the following illustrative drawings.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” include one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference.

General Description

Myostatin (GDF8) has been identified as having important growth-regulatory properties, and in particular, it is known to be a negative regulator of skeletal muscle mass (Kingsley et al. (1994) Genes Dev. 8:133-46; Hoodless, et al. (1998), Curr. Topics Microbiol. Immunol. 228: 235-72; McPheron et al. (1997), Nature 387:83-90; Gonzalez-Cadavid et al. (1998), PNAS, 95:14938-43). Accordingly, due to its negative effects on muscle growth, GDF8 is a therapeutic target for development of compounds and novel therapeutics that can aid in treatment of muscle wasting conditions. In addition to its growth regulating properties, GDF8 is also involved in many other physiological processes, including development of type 2 diabetes and adipose issue disorders, including obesity (Kim et al. (2001) BBRC 281: 902-906). The present invention is based, in part, on the identification of particular truncated polypeptides of the GDF8 and GDF11 prodomain capable of binding and inhibiting at least one of the activities associated with GDF8. The activities of GDF8 refers to one or more growth-regulatory activities associated with an active GDF8 protein, including, but not limited to, inhibition of muscle formation, muscle growth, muscle development, decrease in muscle mass, regulation of muscle enzymes, inhibition of myoblast cell proliferation, modulation of preadipocyte differentiation to adipocytes, increasing sensitivity to insulin, regulation of glucose uptake, glucose homeostasis, and neuronal cell development and maintenance. The invention encompasses myostatin-inhibiting GDF8 and GDF11 prodomain fragments, fusion polypeptides, multimers formed by two or more fusion polypeptides, and nucleic acids which encode these polypeptides and fragments thereof, which comprise a first component P that specifically binds and inhibits myostatin, and a second multimerizing component (M), comprising a component which confers a desired characteristic, such as a longer biological half life, increased stability, the ability to form a higher order structure such as a dimer, and/or the ability to target P to a desired target site, such as skeletal MuSK receptor. Applicants have discovered that by utilizing GDF8 prodomains with deletions in the C terminus of from about 165 amino acids up to about 176 amino acids, or by utilizing GDF11 prodomains with deletions in the C-terminus of from about 173 amino acids up to about 184 amino acids, they can provide ease of production as compared to use of the entire prodomain, as well as decreased antigenicity. Furthermore, the smaller size of these myostatin inhibiting GDF8 or GDF11 polypeptides allows for ease of production for therapeutic purposes, as well as increased stability of the molecule in the bloodstream. In addition, applicants have discovered that in the context of a fusion partner such as an Fc, the peptide-fusions are active and maintain physical stability, thus increasing their half life in the bloodstream. Applicants have further discovered that up to about 29 amino acids of the amino terminus of the GDF8 prodomain and up to about 30 amino acids of the amino terminus of the GDF11 prodomain may also be deleted, along with the carboxy-terminal deletions described, resulting in further production benefits, decreased antigenicity, and ease of producing proteins with multiple domains.

Definitions

The term “GDF8” refers to “Growth and Differentiation Factor 8”, a specific growth and differentiation factor, also known as myostatin. This molecule is a member of the Transforming Growth Factor-beta superfamily of structurally related growth factors. The GDF8 molecule from which the myostatin inhibiting peptides have been derived includes but is not limited to the human homolog, although homologs from other species are contemplated for use. These include but are not limited to bovine, dog, cat, chicken, murine, rat, porcine, ovine, turkey, horse, fish, baboon and other primates. The GDF8 proteins and peptides may be naturally occurring or synthetic. These terms include the full length unprocessed precursor form of the protein, as well as the mature form of the protein resulting from post-translational cleavage of the propeptide. The terms also refer to any fragments or variants of GDF8 that maintain one or more biological activities associated with GDF8 protein, including those described herein, including sequences that have been modified with conservative or non-conservative changes to the amino acid sequence.

The term “GDF11” refers to “Growth and Differentiation Factor 11”, also known as “Bone Morphogenetic Protein 11” or “BMP-11”. This molecule is a member of the Transforming Growth Factor-beta superfamily of structurally related growth factors, all of which possess important growth regulatory and morphogenetic properties (Kinsley et al. (1994), Genes Dev. 8:133-46; Hoodless, et al. (1998), Curr, Topics Microbiol. Immunol. 228:235-72). Various BMP-11 molecules have also been described by McPherron et al. (1997), Proc. Natl. Acad. Sci. USA 94:12457-12461). The GDF11 molecule from which the myostatin inhibiting peptides have been derived includes but is not limited to the human homolog, although homologs from other species are contemplated for use. These include but are not limited to bovine, dog, cat, chicken, murine, rat, porcine, ovine, turkey, horse, fish, baboon and other primates. The GDF11 proteins and peptides may be naturally occurring or synthetic. These terms include the full length unprocessed precursor form of the protein, as well as the mature form of the protein resulting from post-translational cleavage of the propeptide. The terms also refer to any fragments or variants of GDF11 that maintain one or more biological activities associated with GDF11 protein, including those described herein, including sequences that have been modified with conservative or non-conservative changes to the amino acid sequence.

The term “prodomain” is used interchangeably with “propeptide” and refers to the portion of the GDF8 or GDF11 molecule which is cleaved off prior to formation of the mature or active form of GDF8 or GDF11. The organization of the GDF8 and GDF11 molecules is: Signal sequence (SS), followed by “prodomain”, followed by “mature” domain. Thus, after the protein is secreted, there is an amino terminal “prodomain”, followed by the carboxy terminal “mature domain”. The GDF8 or GDF11 prodomain or propeptide is cleaved, leaving carboxy-terminal, mature GDF8 or GDF11. An example of a GDF8 or GDF11 prodomain or propeptide includes, but is not limited to, the sequences set forth in SEQ ID NOs: 1 and 25, respectively. The GDF8 and GDF11 prodomains or propeptides are associated with many functions, including the ability to bind mature GDF8 protein and to inhibit one or more of its activities. The GDF8 and GDF11 prodomains or fragments thereof, as described in the present invention, can act to inhibit at least one or more activities of GDF8. Since GDF8 is a negative regulator of skeletal muscle mass, the GDF8 and GDF11 prodomains or propeptides, or fragments thereof, as described herein, can act to enhance muscle development, muscle mass, muscle formation or muscle cell proliferation. The GDF8 or GDF11 prodomains or fragments thereof may be naturally occurring or synthetic. The GDF8 or GDF11 prodomains and fragments thereof encompass mammalian homologs, most preferably human, although GDF8 and GDF11 prodomain homologs from other sources, including but not limited to, bovine, dog, cat, chicken, murine, rat, porcine, ovine, monkeys and other primates are also included.

The term “fragment” refers to either a protein or polypeptide comprising an amino acid sequence of at least 4 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the amino acid sequence of a parent protein or polypeptide, or a nucleic acid comprising a nucleotide sequence of at least 10 base pairs (preferably at least 20 base pairs, at least 30 base pairs, at least 40 base pairs, at least 50 base pairs, at least 50 base pairs, at least 100 base pairs, at least 200 base pairs) of the nucleotide sequence of the parent nucleic acid. Any given fragment may or may not possess a functional activity of the parent nucleic acid or protein or polypeptide.

Procedures using such conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 ug/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency that may be used are well known in the art.

“Muscle regeneration” and “muscle growth” as used herein refers to the regeneration or growth of muscle, respectively, which may occur by an increase in the fiber size and/or by increasing the number of fibers. The growth of muscle as used herein may be measured by A) an increase in wet weight, B) an increase in protein content, C) an increase in the number of muscle fibers, or D) an increase in muscle fiber diameter. An increase in growth of a muscle fiber can be defined as an increase in the diameter where the diameter is defined as the minor axis of ellipsis of the cross section. The useful therapeutic is one which increases the wet weight, protein content and/or diameter by 10% or more, more preferably by more than 50% and most preferably by more than 100% in a mammal whose muscles have been previously degenerated by at least 10% and relative to a similarly treated control mammal (i.e., a mammal with degenerated muscle tissue which is not treated with the agent that induces muscle growth). A compound or agent, such as a myostatin inhibiting GDF8 or GDF11 prodomain fragment and/or fusion polypeptide and compositions containing these agents, which increase growth by increasing the number of muscle fibers is useful as a therapeutic when it increases the number of fibers in the diseased or atrophied tissue by at least 1%, more preferably at least 20%, and most preferably, by at least 50%. These percentages are determined relative to the basal level in a comparable untreated undiseased mammal or in the contralateral undiseased muscle when the agent is administered and acts locally.

“Atrophy” or “wasting” of muscle as used herein refers to a significant loss in muscle fiber girth. By significant atrophy is meant a reduction of muscle fiber diameter in diseased, injured or unused muscle tissue of at least 10% relative to undiseased, uninjured, or normally utilized tissue.

“Dystrophy” refers to any of several diseases of the muscular system characterized by weakness and wasting of skeletal muscles. There are nine main forms of the disease. They are classified according to the age at onset of symptoms, the pattern of inheritance, and the part of the body primarily affected.

As used herein a polypeptide or peptide “consisting essentially of” or that “consists essentially of” a specified amino acid sequence is a polypeptide or peptide that retains the general characteristics, e.g., activity of the polypeptide or peptide having the specified amino acid sequence and is otherwise identical to that protein in amino acid sequence except it consists of plus or minus 10% or fewer, preferably plus or minus 5% or fewer, and more preferably plus or minus 2.5% or fewer amino acid residues.

The term “about” means within 20%, preferably within 10%, and more preferably within 5%.

A “variant” of a polynucleotide or a polypeptide, as the term is used herein, are polynucleotides or polypeptides that are different from a reference polynucleotide or polypeptide, respectively. Variant polynucleotides are generally limited so that the nucleotide sequence of the reference and the variant are closely related overall and, in many regions, identical. Changes in the nucleotide sequence of the variant may be silent. That is, they may not alter the amino acid sequence encoded by the polynucleotide. Where alterations are limited to silent changes of this type a variant will encode a polypeptide with the same amino acid sequence as the reference. Alternatively, changes in the nucleotide sequence of the variant may alter the amino acid sequence of a polypeptide encoded by the reference polynucleotide. Such nucleotide changes may result in amino acid substitutions, additions, deletions, fusions, and truncations in the polypeptide encoded by the reference sequence. Variant polypeptides are generally limited so that the sequences of the reference and the variant are that are closely similar overall and, in many regions, identical. For example, a variant and reference polypeptide may differ in amino acid sequence by one or more substitutions, additions, deletions, fusions, and truncations, which may be present or absent in any combination. Such variants can differ in their amino acid composition (e.g. as a result of allelic or natural variation in the amino acid sequence, e.g. as a result of alternative mRNA or pre-mRNA processing, e.g. alternative splicing or limited proteolysis) and in addition, or in the alternative, may arise from differential post-translational modification (e.g., glycosylation, acylation, phosphorylation, isoprenylation, lipidation).

Nucleic Acid Construction and Expression of Encoded Fusion Polypeptides

Individual components of the fusion polypeptides of the invention may be produced from nucleic acids molecules using molecular biological methods known to the art. Nucleic acid molecules are inserted into a vector that is able to express the fusion polypeptides when introduced into an appropriate host cell. Appropriate host cells include, but are not limited to, bacterial, yeast, insect, and mammalian cells. Any of the methods known to one skilled in the art for the insertion of DNA fragments into a vector may be used to construct expression vectors encoding the fusion polypeptides of the invention under control of transcriptional/translational control signals. These methods may include in vitro recombinant DNA and synthetic techniques and in vivo recombinations (See Sambrook et al., Molecular Cloning, A Laboratory Manual, Cold Spring Harbor Laboratory; Current Protocols in Molecular Biology, Eds. Ausubel, et al., Greene Publ. Assoc., Wiley-Interscience, NY).

Expression of the nucleic acid molecules of the invention may be regulated by a second nucleic acid sequence so that the molecule is expressed in a host transformed with the recombinant DNA molecule. For example, expression of the nucleic acid molecules of the invention may be controlled by any promoter/enhancer element known in the art.

The nucleic acid constructs of the invention are inserted into an expression vector or viral vector by methods known to the art, wherein the nucleic acid molecule is operatively linked to an expression control sequence. Also provided is a host-vector system for the production of a tissue-specific fusion polypeptide of the invention, which comprises the expression vector of the invention, which has been introduced into a host cell suitable for expression of the fusion polypeptide. The suitable host cell may be a bacterial cell such as E. coli, a yeast cell, such as Pichia pastoris, an insect cell, such as Spodoptera frugiperda, or a mammalian cell, such as a COS, CHO, 293, BHK or NS0 cell.

The invention further encompasses methods for producing the fusion polypeptides of the invention by growing cells transformed with an expression vector under conditions permitting production of the fusion polypeptides and recovery of the fusion polypeptides so produced. Cells may also be transduced with a recombinant virus comprising the nucleic acid construct of the invention.

The fusion polypeptides may be purified by any technique, which allows for the subsequent formation of a stable polypeptide. For example, and not by way of limitation, the fusion polypeptides may be recovered from cells either as soluble polypeptides or as inclusion bodies, from which they may be extracted quantitatively by 8M guanidinium hydrochloride and dialysis. In order to further purify the fusion polypeptides, conventional ion exchange chromatography, hydrophobic interaction chromatography, reverse phase chromatography or gel filtration may be used. The fusion polypeptides may also be recovered from conditioned media following secretion from eukaryotic or prokaryotic cells.

Screening and Detection Methods

The fusion polypeptides of the invention may also be used in in vitro or in vivo screening methods where it is desirable to detect and/or measure target protein levels or, for example, levels of myostatin. Screening methods are well known to the art and include cell-free, cell-based, and animal assays. In vitro assays can be either solid state or soluble. Receptor detection may be achieved in a number of ways known to the art, including the use of a label or detectable group capable of identifying a tissue-specific polypeptide which is bound to a target cell. Detectable labels are well developed in the field of immunoassays and may generally be used in conjunction with assays using the fusion polypeptide of the invention.

A fusion polypeptide of the invention may also be directly or indirectly coupled to a label or detectable group when desirable for the purpose it is being used. A wide variety of labels may be used, depending on the sensitivity required, ease of conjugation, stability requirements, available instrumentation, and disposal provisions. Suitable labels include enzymes such as those discussed below, fluorophores (e.g., fluorescein isothiocyanate (FITC), phycoerythrin (PE), Texas red (TR), rhodamine, free or chelated lanthanide series salts, especially Eu3+, to name a few fluorophores), chromophores, radioisotopes, chelating agents, dyes, colloidal gold, latex particles, ligands (e.g., biotin), and chemiluminescent agents.

Therapeutic Methods

The invention herein further provides for the development of fusion polypeptides described herein as a therapeutic for the treatment of patients suffering from disorders, for example, muscle atrophy. For example, muscle atrophy can result from aging, AIDS-induced cachexia, burns, cancer-induced cachexia, casting, congestive heart failure, settings wherein inflammatory cytokines such as IL-1 or TNF-a are in excess, denervation, diabetes, disuse (such as in prolonged bed rest), growth hormone deficiency, IGF1-deficiency, immobilization, inflammation, such as in chronic inflammatory conditions such as rheumatoid arthritis, mechanic ventilation (resulting in atrophy of the diaphragm), renal failure, sarcopenia, and sepsis-induced cachexia. Denervation may be due to nerve trauma; degenerative; metabolic or inflammatory neuropathy, e.g. Guillian-Barré syndrome; peripheral neuropathy; or nerve damage caused by environmental toxins or drugs. Muscle atrophy may also result from denervation due to a motor neuropathy including, for example, adult motor neuron disease, such as Amyotrophic Lateral Sclerosis (ALS or Lou Gehrig's disease); infantile and juvenile spinal muscular atrophies; and autoimmune motor neuropathy with multifocal conductor block. Muscle atrophy may also result from chronic disease resulting from, for example, paralysis due to stroke or spinal cord injury; skeletal immobilization due to trauma, such as, for example, fracture, ligament or tendon injury, sprain or dislocation; or prolonged bed rest. Metabolic stress or nutritional insufficiency, which may also result in muscle atrophy, include the cachexia of cancer and other chronic illnesses including AIDS, fasting or rhabdomyolysis, and endocrine disorders such as disorders of the thyroid gland and diabetes. Muscle atrophy may also be due to a muscular dystrophy syndromes such as Duchenne, Becker, myotonic, fascioscapulohumeral, Emery-Dreifuss, oculopharyngeal, scapulohumeral, limb girdle, and congenital types, as well as the dystrophy known as Hereditary Distal Myopathy. Muscle atrophy may also be due to a congenital myopathy, such as benign congenital hypotonia, central core disease, nemalene myopathy, and myotubular (centronuclear) myopathy.

Alternatively, the fusion polypeptides of the invention may be used therapeutically in situations where muscle generation or regeneration is desired, or wherein muscle growth or muscle hypertrophy is desired.

Methods of Administration

Methods known in the art for the therapeutic delivery of agents such as proteins or nucleic acids can be used for the therapeutic delivery of fusion polypeptide or a nucleic acid encoding a fusion polypeptide of the invention for treating a deleterious condition or disease in a subject, e.g., cellular transfection, gene therapy, direct administration with a delivery vehicle or pharmaceutically acceptable carrier, indirect delivery by providing recombinant cells comprising a nucleic acid encoding a fusion polypeptide of the invention.

Various delivery systems are known and can be used to administer the fusion polypeptide of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, pulmonary, intranasal, intraocular, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Combination Therapies

In numerous embodiments, the fusion polypeptides of the present invention may be administered in combination with one or more additional compounds or therapies. For example, multiple fusion polypeptides can be co-administered in conjunction with one or more therapeutic compounds. The combination therapy may encompass simultaneous or alternating administration. In addition, the combination may encompass acute or chronic administration.

Pharmaceutical Compositions

The present invention also provides pharmaceutical compositions comprising a fusion polypeptides of the invention and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the fusion polypeptide of the invention which will be effective in the treatment of a condition or disease can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-5000 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Transgenic Animals

The invention includes transgenic non-human animals expressing a fusion polypeptide of the invention. A transgenic animal can be produced by introducing nucleic acid into the male pronuclei of a fertilized oocyte, e.g., by microinjection, retroviral infection, and allowing the oocyte to develop in a pseudopregnant female foster animal. Any of the regulatory or other sequences useful in expression vectors can form part of the transgenic sequence. A tissue-specific regulatory sequence(s) can be operably linked to the transgene to direct expression of the transgene to particular cells. A transgenic non-human animal expressing a fusion polypeptide of the invention is useful in a variety of applications, including as a means of producing such fusion proteins. Further, the transgene may be placed under the control of an inducible promoter such that expression of the tissue-specific fusion polypeptide may be controlled by, for example, administration of a small molecule.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### GDF8 Myostatin Inhibiting Fusion Polypeptide Constructs: Measurement of their Ability to Bind Myostatin

Table 1 illustrates exemplary amino acid and nucleic acid sequences that may be used in the present invention. In particular, exemplary myostatin (GDF8) inhibiting fusion polypeptides (“traps”), are illustrated in SEQ ID NOs: 12, 13, 14, 15, 16, 17, 18, 19 and 20. The following study was done using the myostatin inhibiting fusion polypeptides of SEQ ID NOs 12, 18, and 19, all of which contain various length fragments of the myostatin prodomain, and the results obtained using the myostatin prodomain fragments were compared to the results obtained with a myostatin fusion polypeptide containing the full length myostatin prodomain (SEQ ID NO: 20). The fusion polypeptides were expressed in CHOK1 cells, using a vector which made use of a CMV promoter for gene expression. The myostatin trap plasmid was constructed as follows: using the following oligonucleotide primers a fragment of the myostatin prodomain was generated in a PCR reaction using the full length myostatin cDNA as a template: 5′TTTTTCTCGAGCCCGCCGCCACCATG-CAAAAACTGCAACTC-TGTGTTTATATTTACC (SEQ ID NO: 23) and 3′ AAAAAAAAAAATCCGGA-CCTCTGGACATCA-TACTGATCAATCAGTTC (SEQ ID NO: 24). The PCR fragment was digested with the restriction endonucleases XhoI and BspEI. The fragment was ligated in frame with Fc in a plasmid also digested with XhoI and BspEI. As a negative control empty vector was used to condition CHOK1 media to which purified Fc was added (thus resulting in CHOK1-conditioned media plus purified Fc, to use as a control for the Fc fused to the various myostatin pro domains). The secreted trap or conditioned media was collected. The myostatin trap was compared to SEQ ID NO: 20, which was made using the full length myostatin prodomain fused to Fc (e.g. SEQ ID NO:1 fused to SEQ ID NO:9 with an SG linker), as well as other constructs containing C-terminal deletions of the prodomain, in the following manner: Experiments were conducted to test if the “myostatin traps” could bind myostatin, by determining whether myostatin could be co-immuno-precipitated by each of the potential traps. Thus, an amount of myostatin was added in solution which was equimolar to the putative “myostatin trap” being tested. As negative controls, either myostatin alone or traps alone were also tested. The traps were quantified by Fc ELISA. Myostatin concentration was determined by the manufacturer (R&D). Protein G sepharose beads were used to bind the traps or Fc control, incubating at four degrees Centigrade, overnight. The beads were washed thoroughly with 1 ml of 1% NP40 in PBS 3 times and were boiled in SDS sample buffer containing DTT. The samples were subjected to SDS PAGE and western blotting. Co-immuno-precipitated myostatin was detected using an anti-GDF8 (myostatin) goat polyclonal antibody (R&D Systems), anti goat IgG HRP conjugated secondary antibody and ECL.

The myostatin traps tested (SEQ ID NOs: 12, 18 and 19) were comparable to the myostatin trap made using the full length prodomain of SEQ ID NO: 20 (which is SEQ ID NO: 1 fused to SEQ ID NO: 9 with an SG linker in between) in terms of binding to myostatin.

### Example 2

### GDF8 Myostatin Inhibiting Fusion Polypeptide Constructs: Measurement of their Ability to Block Phosphorylation of SMAD 2 by Myostatin

Myostatin traps of SEQ ID NO: 12, 18 and 19 were expressed in CHOK1 cells, using a vector which made use of a CMV promoter for gene expression. As a negative control empty vector was used to condition CHOK1 media to which purified Fc was added (thus resulting in CHOK1-conditioned media plus purified Fc, to use as a control for the Fc fused to the various myostatin pro domains). From CHOK1 cells transfected with CMV vector expressing the myostatin trap, condtioned media was collected. The amount of trap in the conditioned media was quantified by an ELISA designed to test the Fc portion of the trap. The trap was assessed for its ability to block the activity of myostatin in the following manner: the trap or the Fc control was incubated with myostatin in media and subsequently added to C2C12 myotubes. If myostatin alone is applied to C2C12 cells, it stimulates the phosphorylation of a protein called SMAD2. Therefore, the experiment was designed to test whether the myostatin trap would block the ability of myostatin to induce phosphorylation of SMAD2. Protein extracts were made and subjected to SDS PAGE and western blotting. Phosphorylated and total Smad2 levels were assessed as a measure of myostatin activity using a anti-phospho Smad2(Ser465/467) and anti-Smad2 antibody (Cell Signaling) respectively, anti-rabbit IgG HRP conjugate and ECL to visualize the Western.

The myostatin traps of SEQ ID NO: 12, 18 and 19 were able to block SMAD2 phosphorylation in a manner comparable to a trap made using the full length prodomain of myostatin as shown in SEQ ID NO: 20 (e.g. SEQ ID NO: 1 fused to SEQ ID NO: 9 with an SG linker in between).

TABLE 1
SEQUENCE DESCRIPTIONS for GDF8 inhibitors

| SEQ ID | DESCRIPTION | TYPE |
|---|---|---|
| 1 | Full length human myostatin (GDF 8) pro-domains without signal sequence | Protein |
| 2 | Full length human myostatin (GDF 8) pro-domains without signal sequence | DNA |
| 3 | Amino acids 1-75 of SEQ ID NO: 1 | Protein |
| 4 | Amino acids 1-69 of SEQ ID NO: 1 | Protein |
| 5 | Amino acids 1-64 of SEQ ID NO: 1 | Protein |
| 6 | Amino acids 29-75 of SEQ ID NO: 1 | Protein |
| 7 | Amino acids 29-69 of SEQ ID NO: 1 | Protein |
| 8 | Amino acids 29-64 of SEQ ID NO: 1 | Protein |
| 12 | Amino acids 1-75 of hGDF 8 prodomain fused to hFc with an SG linker | Protein |
| 13 | Amino acids 1-69 of hGDF 8 prodomain fused to hFc with SG linker | Protein |
| 14 | Amino acids 1-64 of hGDF 8 prodomain fused to hFc with an SG linker | Protein |
| 15 | Amino acids 29-75 of hGDF 8 prodomain fused hFc with SG linker | Protein |
| 16 | Amino acids 29-69 of hGDF 8 prodomain fused to hFc with SG linker | Protein |
| 17 | Amino acids 29-64 of hGDF 8 prodomain fused to hFc with an SG linker | Protein |
| 18 | Amino acids 1-103 of hGDF 8 prodomain fused to hFc with SG linker | Protein |
| 19 | Amino acids 1-122 of the hGDF 8 prodomain fused to hFc with SG linker | Protein |
| 20 | Full length hGDF8 fused to hFc with SG linker | Protein |

### Example 3

### GDF11 Myostatin Inhibiting Fusion Polypeptide Constructs: Measurement of their Ability to Bind Myostatin

Table 2 illustrates exemplary amino acid and nucleic acid sequences that may be used in the present invention. In particular, exemplary myostatin inhibiting GDF11 fusion polypeptides, also referred to as GDF11 traps, are illustrated in SEQ ID NOs: 33, 34, 35, 36, 37, 38, and 39. The following protocol demonstrates how one may use the myostatin inhibiting GDF11 fusion polypeptides of SEQ ID NOs 33 through 39 to test their ability to bind myostatin. The fusion constructs containing either the various length fragments of the GDF11 prodomain, or the full length GDF11 prodomain of SEQ ID NO: 25 are tested for myostatin binding. The results obtained using the GDF11 prodomain fragments are compared to the results obtained with a myostatin GDF11 fusion polypeptide containing the full length GDF11 prodomain (SEQ ID NO: 39). The fusion polypeptides are expressed in CHOK1 cells, using a vector which makes use of a CMV promoter for gene expression. The GDF11 trap plasmid is constructed as follows: using the oligonucleotide primers of, a fragment of the GDF11 prodomain is generated in a PCR reaction using the full length GDF11 cDNA as a template: 5′TTTTTCTCGAGCCCGCCGCCACCATGCAAAAACTGCAAC-TCTGTGTTTATATTTACC (SEQ ID NO: 41) and 3′ AAAAAAAAAAATCCGGACCTCTGGACATC-ATACTGATCAATCAGTTC (SEQ ID NO: 42). The PCR fragment is digested with the restriction endonucleases XhoI and BspEI. The fragment is ligated in frame with Fc in a plasmid also digested with XhoI and BspEI. As a negative control empty vector is used to condition CHOK1 media to which purified Fc is added (which results in CHOK1-conditioned media plus purified Fc, to use as a control for the Fc fused to the various GDF11 prodomains). The secreted trap or conditioned media is collected. The GDF11 trap is compared to SEQ ID NO: 39, which is made using the full length GDF11 prodomain fused to Fc (e.g. SEQ ID NO: 25 fused to SEQ ID NO: 9 with an SG linker), as well as other constructs containing C-terminal deletions of the prodomain, in the following manner: Experiments are conducted to test if the “GDF11 traps” could bind myostatin, by determining whether myostatin could be co-immuno-precipitated by each of the potential traps. Thus, an amount of myostatin is added in solution which is equimolar to the putative “GDF11 trap” being tested. As negative controls, either myostatin alone or traps alone are also tested. The traps are quantified by Fc ELISA. Myostatin concentration is determined by the manufacturer (R&D). Protein G sepharose beads are used to bind the traps or Fc control, incubating at four degrees Centigrade, overnight. The beads are washed thoroughly with 1 ml of 1% NP40 in PBS 3 times and are boiled in SDS sample buffer containing DTT. The samples are subjected to SDS PAGE and western blotting. Co-immuno-precipitated myostatin is detected using an anti-GDF8 (myostatin) goat polyclonal antibody (R&D Systems), anti goat IgG HRP conjugated secondary antibody and ECL.

### Example 4

### GDF11 Myostatin Inhibiting Fusion Polypeptide Constructs: Measurement of their Ability to Block Phosphorylation of SMAD 2 by Myostatin

The above-referenced GDF11 traps are expressed in CHOK1 cells, using a vector which makes use of a CMV promoter for gene expression. As a negative control empty vector is used to condition CHOK1 media to which purified Fc is added (thus resulting in CHOK1-conditioned media plus purified Fc, to use as a control for the Fc fused to the various GDF11 prodomains). From CHOK1 cells transfected with CMV vector expressing the GDF11 trap, condtioned media is collected. The amount of trap in the conditioned media is quantified by an ELISA designed to test the Fc portion of the trap. The trap is assessed for its ability to block the activity of myostatin in the following manner: The trap or the Fc control is incubated with myostatin in media and is subsequently added to C2C12 myotubes. If myostatin alone is applied to C2C12 cells, it stimulates the phosphorylation of a protein called SMAD2. Therefore, the experiment is designed to test whether the GDF11 trap would block myostatin's ability to induce phosphorylation of SMAD2. Protein extracts are made and subjected to SDS PAGE and western blotting. Phosphorylated and total Smad2 levels are assessed as a measure of myostatin activity using an anti-phospho Smad2(Ser465/467) and anti-Smad2 antibody (Cell Signaling) respectively, anti-rabbit IgG HRP conjugate and ECL to visualize the Western.

TABLE 2
SEQUENCE DESCRIPTIONS for GDF11 traps

| SEQ ID | DESCRIPTION | TYPE |
|---|---|---|
| 25 | Full length human GDF 11 pro-domain without signal sequence | Protein |
| 26 | Full length human GDF 11 pro-domain without signal sequence | DNA |
| 27 | Amino acids 1-77 of hGDF11 pro-domain | Protein |
| 28 | Amino acids 1-71 of hGDF11 pro-domain | Protein |
| 29 | Amino acids 1-66 of hGDF11 pro-domain | Protein |
| 30 | Amino acids 31-77 of hGDF11 pro-domain | Protein |
| 31 | Amino acids 31-71 of hGDF11 pro-domain | Protein |
| 32 | Amino acids 31-66 of hGDF11 pro-domain | Protein |
| 33 | Amino acids 1-77 of hGDF 11 prodomain fused to hFc with SG linker | Protein |
| 34 | Amino acids 1-71 of hGDF 11 prodomain fused to hFc with SG linker | Protein |
| 35 | Amino acids 1-66 of hGDF 11 prodomain fused to hFc with SG linker | Protein |
| 36 | Amino acids 31-77 of hGDF 11 prodomain fused to hFc with SG linker | Protein |
| 37 | Amino acids 31-71 of hGDF 11 prodomain fused to hFc with SG linker | Protein |
| 38 | Amino acids 31-66 of hGDF 11 prodomain fused to hFc with SG linker | Protein |
| 39 | Full length hGDF11 prodomain fused to hFc with an SG linker | Protein |

### Example 5

### GDF8 Myostatin Inhibiting Fusion Polypeptide Variant Constructs

Additional GDF8 prodomain Fc constructs were generated as described above. Parental hGDF8 traps hGDF8(105)-hFc and hGDF8(1-64)-hFC were modified to convert Cys residues at positions 39, 42, and/or 76 to Ser or Ala. Kinetic and affinity parameters for GDF8 binding by the modified myostatin traps were determined as described above and are summarized in Table 3. Generally, standard Biacore assays were conducted to determine the kinetic and affinity parameters between GDF8 and various myostatin hGDF8 traps. Assay parameters were as follows: Biacore CM5 chip; surface: amine-coupled Protein A; Regeneration: 100 mM H₃PO₄−1×30 s pulse; flow rate: 50 μl/min; sample injection 250 μl; dissociation time: 60 min; rmGDF8 (R&D) concentrations 0.312-20 nM; buffer: HBS-T.

TABLE 3

| | RU | ka | kd | Kd | t½ | Rmax | |
|---|---|---|---|---|---|---|---|
| | captured | (1/Ms) | (1/s) | (pM) | (hr) | (RU) | Chi² |
| hGDF8(1-105)(D76A)-hFc | 139 ± 1.4 | 4.20E⁺⁶ | 5.27E⁻⁵ | 12.5 | 3.65 | 24.5 | 0.436 |
| hGDF8(1-64)-hFc | 147 ± 3 | 1.93E⁺⁶ | 2.50E⁻⁵ | 13.0 | 7.7 | 33.5 | 1.13 |
| hGDF8(1-64)(C39S, C42S)-hFc | 132 ± 2 | 3.50E⁺⁶ | 3.93E⁻⁵ | 11.2 | 4.9 | 37.2 | 0.274 |
| hGDF8(1-38, 43-64)-hFc | 142 ± 3 | 4.66E⁺⁶ | 6.20E⁻⁵ | 13.3 | 3.1 | 24.8 | 0.225 |

What is claimed is:

1. A recombinant nucleic acid molecule encoding a myostatin-inhibiting fusion polypeptide (P)ₓ−M, wherein P is a fragment of growth and differentiation factor (GDF) prodomain GDF8 or GDF11, M is a multimerizing component, and wherein X is a number between 1 and 10.

2. The nucleic acid molecule of claim 2, wherein P is a fragment of GDF8 or GDF11 selected from the group consisting of SEQ ID NO: 3-8 and 27-32.

3. The nucleic acid molecule of claim 1, wherein X is 1, 2 or 3.

4. The nucleic acid molecule of claim 1, wherein M is an immunoglobulin-derived domain selected from the group consisting of an Fc domain of IgG or a heavy chain of IgG.

5. The nucleic acid molecule of claim 1, further comprising a targeting component capable of specifically binding a muscle surface protein.

6. The nucleic acid molecule of claim 8, wherein the targeting component comprises an N-terminal extracellular domain of muscle cadherin or a MuSK receptor.

7. The nucleic acid molecule of claim 6, wherein the targeting protein is agrin or a fragment or derivative thereof capable of binding MuSK.

8. A vector comprising the nucleic acid molecule of claim 1.

9. A myostatin-inhibiting fusion polypeptide encoded by the nucleic acid molecule of claim 1.

10. The myostatin inhibiting fusion polypeptide of claim 9 selected from the group consisting of SEQ ID NO:12-20 and 33-39.

11. A vector comprising the nucleic acid molecule of claim 1.

12. A host-vector system for producing a myostatin inhibiting fusion polypeptide, comprising the vector of claim 11 in a suitable host cell.

13. The host-vector system of claim 12, wherein the suitable host cell is selected from the group consisting of a bacterial, yeast, insect, and a mammalian cell.

14. The host-vector system of claim 13, wherein the cell is selected from the group consisting of an E. coli, a B. subtilis, a BHK, a COS and a CHO cell.

15. A method of producing a myostatin-inhibiting fusion polypeptide, comprising culturing a host cell transfected with the vector of claim 11, under conditions suitable for expressing the polypeptide from the host cell, and recovering the polypeptide so produced.

16. A myostatin-inhibiting fusion polypeptide (P)ₓ−M, wherein P is a fragment of a growth and differentiation factor (GDF) prodomain GDF8 or GDF11, M is a multimerizing component, and wherein X is a number between 1 and 10.

17. A dimeric molecule comprising the fusion polypeptide of claim 16.

18. A pharmaceutical composition comprising the dimeric molecule of claim 17 and a pharmaceutically acceptable carrier.

19. A method of treating muscle atrophy, inducing muscle growth, hypertrophy, or regeneration comprising administering the pharmaceutical composition of claim 18 to a subject in need thereof.

20. The method of claim 19, wherein the muscle atrophy results from a condition selected from the group consisting of aging, denervation, casting, inactivity, bed rest, congestive heart failure, diabetes, renal failure, growth hormone deficiency, IGF1-deficiency, immobilization, inflammation, such as in chronic inflammatory conditions such as rheumatoid arthritis, mechanic ventilation, renal failure, sarcopenia, sepsis-induced cachexia, glucocorticoid-induced atrophy, cytokine-induced atrophy settings, sepsis-induced atrophy, cachexia associated with AIDS, cachexia associated with cancer, cachexia associated with burns, degenerative neuropathy, metabolic neuropathy, inflammatory neuropathy, spinal muscular atrophy, autoimmune motor neuropathy, acute myocardial infarction, glucocorticoid-induced osteoporosis, obesity and muscular dystrophy.


SEQUENCE LISTING (42 sequences)

SEQ ID NO: 1 - protein, 239 aa, Homo sapiens
NENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPK

SEQ ID NO: 2 - DNA, 717 bp, Homo sapiens
aatgagaacagtgagcaaaaagaaaatgtggaaaaagaggggctgtgtaatgcatgtacttggagacaaaacactaaatcttcaagaatagaagccattaagatacaaatcctcagtaaacttcgtctggaaacagctcctaacatcagcaaagatgttataagacaacttttacccaaagctcctccactccgggaactgattgatcagtatgatgtccagagggatgacagcagcgatggctctttggaagatgacgattatcacgctacaacggaaacaatcattaccatgcctacagagtctgattttctaatgcaagtggatggaaaacccaaatgttgcttctttaaatttagctctaaaatacaatacaataaagtagtaaaggcccaactatggatatatttgagacccgtcgagactcctacaacagtgtttgtgcaaatcctgagactcatcaaacctatgaaagacggtacaaggtatactggaatccgatctctgaaacttgacatgaacccaggcactggtatttggcagagcattgatgtgaagacagtgttgcaaaattggctcaaacaacctgaatccaacttaggcattgaaataaaagctttagatgagaatggtcatgatcttgctgtaaccttcccaggaccaggagaagatgggctgaatccgtttttagaggtcaaggtaacagacacaccaaaa

SEQ ID NO: 3 - protein, 75 aa, Homo sapiens
  carboxy terminal deletion of human myostatin prodomain
NENSEQKENVEKEGLCNACTWRQNTKSSRIEAIKIQILSKLRLETAPNISKDVIRQLLPKAPPLRELIDQYDVQR

SEQ ID NO: 4 - protein, 69 aa, Homo sapiens
  carboxy terminal deletion of human myostatin prodomain
NENSEQKENVEKEGLCNACTWRQNTKSSRIEAIKIQILSKLRLETAPNISKDVIRQLLPKAPPLRELID

SEQ ID NO: 5 - protein, 64 aa, Homo sapiens
  carboxy terminal deletion of human myostatin prodomain
NENSEQKENVEKEGLCNACTWRQNTKSSRIEAIKIQILSKLRLETAPNISKDVIRQLLPKAPPL

SEQ ID NO: 6 - protein, 47 aa, Homo sapiens
  amino and carboxy terminal deletion of human myostatin prodomain
RIEAIKIQILSKLRLETAPNISKDVIRQLLPKAPPLRELIDQYDVQR

SEQ ID NO: 7 - protein, 41 aa, Homo sapiens
  amino and carboxy terminal deletion of human myostatin prodomain
RIEAIKIQILSKLRLETAPNISKDVIRQLLPKAPPLRELID

SEQ ID NO: 8 - protein, 36 aa, Homo sapiens
  amino and carboxy terminal deletion of human myostatin prodomain
RIEAIKIQILSKLRLETAPNISKDVIRQLLPKAPPL

SEQ ID NO: 9 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 681 bp, Homo sapiens
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 11 - protein, 418 aa, Homo sapiens
PLVQIKSDKQQLGSVIYSIQGPGVDEEPRGVFSIDKFTGKVFLNAMLDREKTDRFRLRAFALDLGGSTLEDPTDLEIVVVDQNDNRPAFLQEAFTGRVLEGAVPGTYVTRAEATDADDPETDNAALRFSILQQGSPELFSIDELTGEIRTVQVGLDREVVAVYNLTLQVADMSGDGLTATASAIITLDDINDNAPEFTRDEFFMEAIEAVSGVDVGRLEVEDRDLPGSPNWVARFTILEGDPDGQFTIRTDPKTNEGVLSIVKALDYESCEHYELKVSVQNEAPLQAAALRAERGQAKVRVHVQDTNEPPVFQENPLRTSLAEGAPPGTLVATFSARDPDTEQLQRLSYSKDYDPEDWLQVDAATGRIQTQHVLSPASPFLKGGWYRAIVLAQDDASQPRTATGTLSIEILEVNDHAP

SEQ ID NO: 12 - protein, 304 aa, Homo sapiens
NENSEQKENVEKEGLCNACTWRQNTKSSRIEAIKIQILSKLRLETAPNISKDVIRQLLPKAPPLRELIDQYDVQRSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 298 aa, Homo sapiens
NENSEQKENVEKEGLCNACTWRQNTKSSRIEAIKIQILSKLRLETAPNISKDVIRQLLPKAPPLRELIDSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 293 aa, Homo sapiens
NENSEQKENVEKEGLCNACTWRQNTKSSRIEAIKIQILSKLRLETAPNISKDVIRQLLPKAPPLSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 276 aa, Homo sapiens
RIEAIKIQILSKLRLETAPNISKDVIRQLLPKAPPLRELIDQYDVQRSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 270 aa, Homo sapiens
RIEAIKIQILSKLRLETAPNISKDVIRQLLPKAPPLRELIDSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 265 aa, Homo sapiens
RIEAIKIQILSKLRLETAPNISKDVIRQLLPKAPPLSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 332 aa, Homo sapiens
NENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 351 aa, Homo sapiens
NENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 468 aa, Homo sapiens
NENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 456 aa, Homo sapiens
MSALLILALVGAAVADYKDDDDKQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPKSRKVPHTVLNLKEPLYVGGAPDFSKLARAAAVSSGFDGAIQLVSLGGRQLLTPEHVLRQVDVTSFAGHPCTRASGHPCLNGASCVPREAAYVCLCPGGFSGPHCEKGLVEKSAGDVDTLAFDGRTFVEYLNAVTESELANEIPVEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 22 - protein, 23 aa, Homo sapiens
MQKLQLCVYIYLFMLIVAGPVDL

SEQ ID NO: 23 - DNA, 57 bp, Artificial Sequence
  primer
tttttctcgagcccgccgccaccatgcaaaaactgcaactctgtgtttatatttacc

SEQ ID NO: 24 - DNA, 47 bp, Artificial Sequence
  primer
cctgactaactagtcatactacaggtctccaggcctaaaaaaaaaaa

SEQ ID NO: 25 - protein, 250 aa, Homo sapiens
GERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTK

SEQ ID NO: 26 - DNA, 750 bp, Homo sapiens
ggggagcgctccagccggccagccccgtccgtggcgcccgagccggacggctgccccgtgtgcgtttggcggcagcacagccgcgagctgcgcctagagagcatcaagtcgcagatcttgagcaaactgcggctcaaggaggcgcccaacatcagccgcgaggtggtgaagcagctgctgcccaaggcgccgccgctgcagcagatcctggacctacacgacttccagggcgacgcgctgcagcccgaggacttcctggaggaggacgagtaccacgccaccaccgagaccgtcattagcatggcccaggagacggacccagcagtacagacagatggcagccctctctgctgccattttcacttcagccccaaggtgatgttcacaaaggtactgaaggcccagctgtgggtgtacctacggcctgtaccccgcccagccacagtctacctgcagatcttgcgactaaaacccctaactggggaagggaccgcagggggagggggcggaggccggcgtcacatccgtatccgctcactgaagattgagctgcactcacgctcaggccattggcagagcatcgacttcaagcaagtgctacacagctggttccgccagccacagagcaactggggcatcgagatcaacgcctttgatcccagtggcacagacctggctgtcacctccctggggccgggagccgaggggctgcatccattcatggagcttcgagtcctagagaacacaaaa

SEQ ID NO: 27 - protein, 77 aa, Homo sapiens
  carboxy terminal deletion
GERSSRPAPSVAPEPDGCPVCVWRQHSRELRLESIKSQILSKLRLKEAPNISREVVKQLLPKAPPLQQILDLHDFQG

SEQ ID NO: 28 - protein, 71 aa, Homo sapiens
  carboxy terminal deletion
GERSSRPAPSVAPEPDGCPVCVWRQHSRELRLESIKSQILSKLRLKEAPNISREVVKQLLPKAPPLQQILD

SEQ ID NO: 29 - protein, 66 aa, Homo sapiens
  carboxy terminal deletion
GERSSRPAPSVAPEPDGCPVCVWRQHSRELRLESIKSQILSKLRLKEAPNISREVVKQLLPKAPPL

SEQ ID NO: 30 - protein, 47 aa, Homo sapiens
  carboxy and amino terminal deletion
RLESIKSQILSKLRLKEAPNISREVVKQLLPKAPPLQQILDLHDFQG

SEQ ID NO: 31 - protein, 41 aa, Homo sapiens
  carboxy and amino terminal deletion
RLESIKSQILSKLRLKEAPNISREVVKQLLPKAPPLQQILD

SEQ ID NO: 32 - protein, 36 aa, Homo sapiens
  carboxy and amino terminal deletion
RLESIKSQILSKLRLKEAPNISREVVKQLLPKAPPL

SEQ ID NO: 33 - protein, 306 aa, Homo sapiens
GERSSRPAPSVAPEPDGCPVCVWRQHSRELRLESIKSQILSKLRLKEAPNISREVVKQLLPKAPPLQQILDLHDFQGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 300 aa, Homo sapiens
GERSSRPAPSVAPEPDGCPVCVWRQHSRELRLESIKSQILSKLRLKEAPNISREVVKQLLPKAPPLQQILDSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 295 aa, Homo sapiens
GERSSRPAPSVAPEPDGCPVCVWRQHSRELRLESIKSQILSKLRLKEAPNISREVVKQLLPKAPPLSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 276 aa, Homo sapiens
RLESIKSQILSKLRLKEAPNISREVVKQLLPKAPPLQQILDLHDFQGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 270 aa, Homo sapiens
RLESIKSQILSKLRLKEAPNISREVVKQLLPKAPPLQQILDSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 265 aa, Homo sapiens
RLESIKSQILSKLRLKEAPNISREVVKQLLPKAPPLSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 479 aa, Homo sapiens
GERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 23 aa, Artificial Sequence
  signal polypeptide
MQKLQLCVYIYLFMLIVAGPVDL

SEQ ID NO: 41 - DNA, 57 bp, Artificial Sequence; identical to SEQ ID NO: 23
  primer

SEQ ID NO: 42 - DNA, 47 bp, Artificial Sequence
  primer
cttgactaactagtcatactacaggtctccaggcctaaaaaaaaaaa